(19) United States
(12) Patent Application Publication
(10) Pub. No.: US 2026/0254933 A1
(43) Pub. Date: Aug. 27, 2026

(54) Imaging Device, Imaging System, and Method for Operating an Imaging Device

(71) Applicant: KARL STORZ SE & Co. KG, Tuttlingen (DE)

(72) Inventors: Sebastian Barth, Neuhausen ob Eck (DE); Franz Bauer, Tuttlingen (DE)

(73) Assignee: KARL STORZ SE & Co. KG, Tuttlingen (DE)

(21) Appl. No.: 19/419,267

(22) Filed: Dec. 15, 2025

(30) Foreign Application Priority Data
Dec. 20, 2024 (DE) 10 2024 139 198.7

(51) Int. Cl. H04N 13/207; H04N 23/50; H04N 23/55; H04N 23/695

(52) U.S. Cl. CPC H04N 13/207; H04N 23/55; H04N 23/555; H04N 23/695

(57) ABSTRACT

The invention relates to an imaging device (10; 110; 210), in particular an endoscope device, comprising a shaft (12; 112; 212) that defines a longitudinal axis (14; 114; 214), and comprising a stereoscopy unit (16; 116; 216) for stereoscopic image generation. It is proposed that the stereoscopy unit (16; 116; 216) comprises a movement unit (20; 120; 220) for a mechanically effected change to a selection region (22, 22'; 122, 122'; 222, 222') from a field of view (24; 124; 224) of the stereoscopy unit (16; 116; 216) for image capture, wherein the stereoscopy unit (16; 116; 216) is configured to define the selection region (22, 22'; 122, 122'; 222, 222') for image capture for use in stereoscopic image generation as a function of a rotation parameter with respect to the longitudinal axis (14; 114; 214). Fig. 2

### TECHNICAL FIELD OF THE INVENTION

The present invention relates to an imaging device for stereoscopic image generation, comprising a shaft that defines a longitudinal axis, and a stereoscopy unit for stereoscopic image generation. Furthermore, the present invention relates to an imaging system having such an imaging device and a method for operating such an imaging device.

### BACKGROUND

The development of modern 3D endoscopes places high demands on the precise alignment of their optical systems. From the prior art, for example from US 2012/0188347 A1, stereoscopic devices comprising a single imaging path with an associated field of view are known. US 2012/0188347 A1 teaches various configuration options for recording images from different perspectives from different regions of the field of view.

Furthermore, from the prior art, endoscopes are known that use two spatially separated optical systems for stereoscopic image generation in order to capture an object point from different viewing angles. With these endoscopes having two optical systems, the two optical systems must always be aligned in a horizontal position to realistically replicate human depth perception.

However, a problem arises in that rotation of the endoscope can lead to the horizontal alignment no longer being maintained, as a result of which the eye plane of the user no longer coincides with the plane of the two optical systems and the 3D impression is lost. This applies in particular to devices that only use a single imaging path.

A solution approach known, for example, from US 10,365,554 B1 provides a stereo endoscope comprising a liquid crystal layer. By means of the liquid crystal layer, aperture openings for stereoscopic image capture can be electronically defined. Upon rotation of the endoscope, the alignment with respect to a reference alignment of the endoscope is determined, and based thereon the positions of the aperture openings are electronically adjusted. Such electronic aperture openings require a complex control environment to accurately capture and control the opening positions. This complexity increases development effort and production costs. Factors such as temperature fluctuations, electromagnetic interference (EMI) and material-related properties can negatively affect performance.

The object of the present invention is to provide an imaging device having a particularly compact structure and simplified construction, which at least partially solves the aforementioned problems according to the prior art and advantageously ensures a three-dimensional representation in different viewing directions of the imaging device.

### SUMMARY OF THE INVENTION

This object is achieved according to the invention by a device and a method having the features of claims 1, 14 and 15. Advantageous configurations and developments of the invention can be found in the dependent claims.

According to the invention, an imaging device, in particular an endoscope device, is provided for stereoscopic image generation. The imaging device comprises a shaft that defines a longitudinal axis and a stereoscopy unit for stereoscopic image generation. The stereoscopy unit comprises a movement unit for a mechanically effected change to a selection region from a field of view of the stereoscopy unit for image capture. The stereoscopy unit is further configured to define the selection region for capturing images for use in stereoscopic image generation as a function of a rotation parameter with respect to the longitudinal axis.

By being able to mechanically change the selection region and define it as a function of a rotation parameter with respect to the longitudinal axis, the alignment of selection regions used for capturing images in stereoscopic image generation can be kept horizontal relative to one another. An expedient stereoscopic image generation can thus be made possible, for example, at different rotation angles of the shaft and/or the stereoscopy unit about the longitudinal axis. In addition, the mechanically effected change to the selection region eliminates the need to use two separate optical systems. The use of only a single optical system, in particular a single imaging path, is sufficient for expedient stereoscopic image generation, in particular even at different rotation angles of the shaft, by the imaging device according to the invention. An imaging device with simplified control can thus be provided. This allows a particularly reliable and responsive adaptation of the stereoscopic image generation to rotations of the imaging device.

The imaging device can be designed as an endoscope device, which can be designed as part of an endoscope or can comprise the entire endoscope. Alternatively, the imaging device can also be designed as an exoscope device, in particular as part of an exoscope, or comprise the entire exoscope. The imaging device is designed for stereoscopic image generation, for example as a stereoendoscope or stereoexoscope. The imaging device can be configured to capture images of an object point from different perspectives in order to make stereoscopic image generation possible. The imaging device can be intended for medical or non-medical applications.

The stereoscopy unit can be configured for stereoscopic image generation. In particular, the stereoscopy unit can at least be configured to capture the images required for stereoscopic image generation. The stereoscopy unit can comprise a sensor unit for capturing the images required for stereoscopic image generation. The stereoscopy unit can comprise an optical unit for guiding light rays, in particular from an object to be captured to the sensor unit. The stereoscopy unit can comprise a control unit that can be configured to control the capture of images for use in stereoscopic image generation, for example, a capture time and/or readout time via the sensor unit. The control unit can be configured to control the movement unit. In addition, the control unit can be configured to combine the images captured by means of the sensor unit for stereoscopic image generation. Alternatively, a separate computing unit can be provided for the control unit, which can be configured to combine the images captured by the sensor unit for stereoscopic image generation.

The stereoscopy unit can be configured to generate different perspectives for capturing images of an examination object for stereoscopic image generation, in particular by defining different selection regions. In other words, the stereoscopy unit for stereoscopic image generation can be configured to capture images with different and mechanically changed selection regions in a rotation position with respect to the longitudinal axis. Different selection regions can be understood to mean selection regions comprising different positions, in particular with respect to the longitudinal axis.

The optical unit can comprise an optical viewing direction unit that defines a viewing direction for observing an examination object. The viewing direction unit can be arranged at a distal end of the shaft. An arrangement of the viewing direction unit at the distal end of the shaft can correspond to an arrangement in a distal end region of the shaft. In other words, the viewing direction unit does not have to form the distal tip of the shaft. Distal to the viewing direction unit, a further optical element, such as a lens optic, a protective glass or the like, can be arranged. The viewing direction unit can have one or more reflective flat or curved surfaces, for example inside or on a prism or between a plurality of prisms.

Within the scope of the present disclosure, a proximal portion or proximal side is a portion or side that is arranged closer to the observer/user and further away from the field of view/patient than a distal portion or distal side. Similarly, a distal portion or distal side is a portion or side that is arranged closer to the field of view/patient and further away from the observer/user than a proximal portion or proximal side. Accordingly, distal can also be described as close to the patient, facing the patient, and/or distant from the observer. Proximal can also be described as distant from the patient, facing away from the patient, and/or close to the observer. When used as an endoscopic instrument, the distal end of the shaft is usually inserted into the body in order to allow observations to be made there. At least the proximal end of the instrument protrudes from the body because this is where the operator handles and controls it.

The field of view can be defined by the optical unit, in particular by the viewing direction unit. For the purposes of explaining the present invention, the term "field of view of the stereoscopy unit" is to be understood to mean the maximum visible region through the optical unit with respect to a viewing direction. In other words, the field of view can correspond to a totality of possible selection regions. The selection region can represent a portion of the field of view. The selection region can define a region, in particular a perspective, that can be captured by the stereoscopy unit in order to capture an image for stereoscopic image generation. The region determined by the selection region can thus define an imaging region or image capture region. For stereoscopic image generation, the stereoscopy unit can be configured to capture images with two different selection regions, i.e. from two different perspectives, which can be combined to form a stereoscopic image. The selection region can function as a kind of aperture or aperture cutout.

The movement unit can be configured to change the position of the selection region. The selection region can be changed by the movement unit in a selection plane. The selection plane can run perpendicular to the longitudinal axis or be arranged at an angle greater than 90° and less than 180° relative to the longitudinal axis. The field of view can correspond to a totality of possible selection regions in the selection plane. The selection plane can run at an angle of 90° to 180° to the viewing direction, in particular depending on the nature or setting of the viewing direction unit. Additionally, the movement unit can be configured to change the size and/or shape of the selection region. Alternatively, the selection region can be unchangeable with respect to its size and/or shape.

For the purposes of explaining the present invention, a "mechanically effected change" of a selection region is to be understood to mean a change in at least one position of the selection region in the selection plane and/or relative to the longitudinal axis by a mechanical movement of the selection region. In particular, the mechanically effected change can alter the rotation position of the selection region about the longitudinal axis, wherein the stereoscopy unit defines the rotation position as a function of a rotation parameter with respect to the longitudinal axis. The movement unit can, for example, have active and/or passive movement mechanisms. For example, the movement unit can comprise a drive, in a particular hydraulic, electric, piezoelectric or pneumatic drive, for mechanically effecting a change in the selection region. A passive movement mechanism can, for example, be a movement mechanism based on spring tension, gravity, elasticity or the like.

The control unit can be configured to define the capture time and/or readout time via the sensor unit for capturing an image for stereoscopic image generation, as a function of the position of the selection region. The control unit can be configured to define the position for the selection region and/or the capture time and/or readout time via the sensor unit for capturing an image for stereoscopic image generation, as a function of the rotation parameter with respect to the longitudinal axis. The rotation parameter can, for example, describe a rotation angle of the shaft and/or the stereoscopy unit, or of the viewing direction unit, about the longitudinal axis. The rotation angle can be determined, for example, in relation to an initial position of the shaft. The initial position can be determined, for example, by the initial orientation of the stereoscopy unit or of the viewing direction unit. For example, a horizontal position of the viewing direction of the stereoscopy unit can be selected as the initial position. In principle, the initial position can be selected arbitrarily; in order to define the selection region for capturing images, only a change relative to the initial position is relevant, for example, a change in the rotation angle of the shaft and/or the stereoscopy unit, or of the viewing direction unit.

According to a preferred exemplary embodiment, the stereoscopy unit can comprise an aperture unit comprising at least one mechanically positionable aperture opening, wherein the stereoscopy unit is configured to determine a position of the aperture opening for capturing images for use in stereoscopic image generation as a function of a rotation parameter, in particular the one already mentioned, with respect to the longitudinal axis. In a preferred exemplary embodiment of the invention, it can be provided that the aperture unit comprises only a single aperture opening. By using an aperture unit with one aperture opening, a stereoscopy unit with a particularly simple design with respect to its control requirements can be provided. The selection region can be defined by the aperture opening, in particular by the position of the aperture opening. The aperture unit can comprise a disk or the like, which can comprise the aperture opening. The aperture unit can be arranged to be movable, in particular rotatable, with respect to the shaft. The field of view of the stereoscopy unit can be defined by the totality of possible arrangement positions of the aperture opening. Accordingly, a selection region is determined by the positioning of the aperture opening in front of the field of view. The opening region of the aperture opening is thus smaller than the field of view of the stereoscopy unit and can determine the selection region. By configuring the stereoscopy unit with an aperture unit that comprises a mechanically positionable aperture opening, a stereoscopy unit that is particularly robust and requires particularly little effort with respect to control can be provided.

In one embodiment variant of the imaging device, the movement unit comprises, for example, a planar drive for translating the aperture opening in a plane perpendicular to the longitudinal axis. The planar drive is advantageously configured in such a way that it positions the aperture opening as a function of the rotation parameter. The position of the aperture opening is determined as a function of the rotation angle of the endoscope. The planar drive, for example, is a piezoelectric drive with an X-actuator and a Y-actuator.

In addition to a solution in which the stereoscopy unit comprises an aperture unit, the stereoscopy unit can also comprise a reflection unit comprising mechanically alignable, in particular tiltable relative to the longitudinal axis, reflection elements, wherein the stereoscopy unit can be configured to determine an alignment of the reflection elements for capturing images for use in stereoscopic image generation as a function of a rotation parameter with respect to the longitudinal axis, in particular the one already mentioned above. The design of the stereoscopy unit comprising a reflection unit makes a particularly flexible design of the stereoscopy unit possible. The alignment of the reflection elements can define the selection region. In particular, a maximum reflective surface of the reflection unit, which can be defined by the reflection elements, can correspond to the field of view of the stereoscopy unit. The selection region can be defined by the reflection elements that reflect onto the sensor unit light rays of an examination object to be captured. The reflection unit can, for example, be designed as a micro-electromechanical system (MEMS). The reflection elements can be designed as mirrors, in particular MEMS mirrors, as prisms, as diffraction grating or the like.

The aperture opening of the aperture unit and/or the reflection element of the reflection unit can be understood to mean a kind of selection means or selection element of the movement unit for selecting a selection region from the field of view of the stereoscopy unit. By mechanically changing a position, in particular a circumferential position or angular setting, of the selection means or selection element relative to the longitudinal axis, different selection regions can be selected. According to the invention, the selection means or selection element is determined as a function of a rotation parameter with respect to the longitudinal axis, such as a rotation angle of the shaft and/or the stereoscopy unit or of the viewing direction unit about the longitudinal axis, as described above. For example, the rotation position of the stereoscopy unit, the angular velocity of the aperture opening and/or a rotation position of the aperture opening can also serve as rotation parameters for defining the selection regions.

According to a further development of the invention, the stereoscopy unit can be configured to capture at least two images at different selection regions for stereoscopic image generation, in particular at different positions of the aperture opening or at different alignments of the reflection elements. The stereoscopy unit can in particular be configured to capture two images with different selection regions, which represent two different perspectives and can be combined for stereoscopic image generation. The two images can be captured at different selection regions, in particular with different positions of the aperture opening or different alignments of the reflection elements. The configuration according to the invention of the stereoscopy unit makes possible the capture of images of an examination object from different perspectives for stereoscopic image generation in a particularly compact manner.

Within the scope of the invention, it can be provided that the stereoscopy unit is configured to keep an alignment of a stereo base, defined by the selection region or the two selection regions, constant during rotations about the longitudinal axis. The alignment of the stereo base can thus be defined in particular by the positioning of the aperture opening or the alignment of the reflection elements when capturing images for stereoscopic image generation and can be kept constant during rotations about the longitudinal axis by defining the selection region as a function of the rotation parameter with respect to the longitudinal axis. It can thus be achieved that, for example, during rotations of the shaft and/or of the stereoscopy unit, or of the viewing direction unit, about the longitudinal axis, the quality of the stereoscopic image generation can be maintained.

The stereo base can be defined by the positions of the selection regions, in particular the positions of at least one aperture opening or the positions of the capture region defined by the alignment of the reflection elements, at which the images are captured for stereoscopic image generation. The stereo base can be determined in particular by a connecting line between the two selection regions, that is to say, by a connecting line between the two positions for the at least one aperture opening or the positions of the capture regions defined by the alignment of the reflection elements. The stereoscopy unit can be configured to determine the capture time and/or readout time via the sensor unit for capturing the images for stereoscopic image generation and/or the positions of the selection regions in such a way that the stereo base is constant and can be kept in a horizontal position. For the purposes of explaining the invention, the statement that the stereo base is maintained in a horizontal position is to be understood to mean that a parallax between the two selection regions runs horizontally.

According to a development of the invention, it can be provided that the stereoscopy unit comprises only a single imaging path for receiving the images for stereoscopic image generation. Due to the configuration according to the invention of the stereoscopy unit, it is not necessary to use two separate optical systems, that is to say, imaging paths. The use of a single imaging path is sufficient for expedient stereoscopic image generation at different rotation angles of the shaft and/or the viewing direction unit with respect to the longitudinal axis. Thus, it is possible to provide an imaging device that exhibits a particularly simple and space-saving design. This helps to minimize a number of manufacturing and assembly-related error sources. The imaging device can comprise only one optical system, which may be configured to transmit light from the objective lens and/or the viewing direction unit toward a proximal end of the imaging device, preferably toward the sensor unit. The optical system can be designed as a rod lens system or as another relay lens system. The optical system can be part of the optical unit.

It can be provided that the stereoscopy unit comprises a position sensor system for determining the rotation parameter, in particular the rotation angle of the shaft and/or the stereoscopy unit, or the viewing direction unit, about the longitudinal axis. The stereoscopy unit can be configured to define the selection region for capturing images for use in stereoscopic image generation, as a function of the rotation parameter with respect to the longitudinal axis captured by means of the position sensor system. In particular, the stereoscopy unit can be configured to define at least one capture time and/or readout time via the sensor unit for the capture of an image for stereoscopic image generation, as a function of the rotation parameter captured by means of the position sensor system. For example, the control unit can control the sensor unit in such a way that it captures an image of the associated selection region at the capture time and/or readout time. Additionally or alternatively, the control unit can be configured to control the movement unit as a function of the rotation parameter captured by means of the position sensor system. It can also be provided that the movement unit is configured to continuously and periodically change the selection region and the stereoscopy unit merely adapts a capture time and/or readout time via the sensor unit as a function of the rotation parameter. The position sensor system can comprise a gyroscope, an accelerometer, an inertial measurement unit, a combination of these or similar devices. The position sensor system can be connected to the control unit, at least in terms of data technology.

According to a further development of the invention, it can be provided that the longitudinal axis and the viewing direction of the viewing direction unit enclose an acute angle greater than zero. The angle between the longitudinal axis and the viewing direction can be referred to as the viewing angle. The imaging device can be designed as an oblique-viewing endoscope, or the endoscope can be configured as an oblique-viewing endoscope by the imaging device. Due to the configuration of the stereoscopy unit according to the invention, a rotation of the shaft and/or the stereoscopy unit or the viewing direction unit about the longitudinal axis to change the viewing direction can be compensated for by adapting the selection region or the selection regions for capturing images for stereoscopic image generation as a function of the rotation about the longitudinal axis in order to achieve a horizontal alignment of the stereoscopic base.

In a preferred exemplary embodiment of the invention, it can be provided that the aperture unit comprises an aperture element comprising the aperture opening, which is rotatably arranged about a rotation axis. Due to such an aperture unit, a stereoscopy unit can be provided that is particularly robust and requires particularly little effort with respect to control. The aperture element can be designed as the previously mentioned disk. According to a further development of the invention, the rotation axis can be aligned parallel to the longitudinal axis. In particular, the rotation axis can lie on the longitudinal axis. It can be provided that the aperture opening is arranged eccentrically with respect to the rotation axis.

In a preferred exemplary embodiment, the movement unit can be configured to drive the aperture element to rotate about the rotation axis at a constant angular velocity. The movement unit can, for example, comprise a rotation actuator or the like to drive the aperture element to rotate about the rotation axis at a constant angular velocity. The movement unit can be configured to continuously drive the aperture element at a constant angular velocity during operation of the imaging device. For example, the stereoscopy unit is configured to define a capture time and/or readout time via the sensor unit for capturing the images for stereoscopic image generation in such a way that the stereo base determined by the corresponding positions of the aperture opening is located in the horizontal position. It may be advantageous to provide that the positions of the aperture opening, i.e. the positioning of the selection regions where the images are captured for stereoscopic image generation, are arranged opposite one another with respect to the longitudinal axis.

It can be provided that the reflection unit is rotatably arranged about an axis of the optical imaging path, in particular about the longitudinal axis. The movement unit can be configured to rotate the reflection unit, in particular the reflection elements, about an angle about the longitudinal axis that corresponds in magnitude to a rotation angle of the shaft and/or the viewing direction unit about the longitudinal axis and is in particular opposite to this rotation angle. Alternatively, the reflection unit can be arranged in a rotationally fixed manner relative to the longitudinal axis and/or the viewing direction unit, wherein the reflection elements can be aligned with respect to the longitudinal axis. In particular, the reflection elements can be mounted in the reflection unit in a tiltable manner, in order to be alignable with respect to the longitudinal axis. Such a configuration of the stereoscopy unit allows for a particularly flexible design of the imaging device, which simultaneously enables stereoscopic image generation at different rotation angles of the shaft and/or the viewing direction unit about the longitudinal axis. For example, the angular position of the reflection unit or reflection element, or a tilt angle as a function of the rotation position of the shaft or stereoscopy unit, can be used to define the selection regions.

The invention further relates to an imaging system having the imaging device according to the invention as described above. The imaging system can be designed as an endoscopy system or as an exoscopy system. An imaging system can thus be provided which reliably makes stereoscopic image generation possible even when the viewing direction changes. Due to the use of the imaging device according to the invention in the imaging system, a particularly compact system can be provided. The imaging system can have a display. The imaging system can comprise a computing unit for combining the images captured by means of the stereoscopy unit. The computing unit can comprise at least one processor and one memory element, along with an operating program stored on the memory element. The memory element can be designed as a digital storage medium, for example as a memory chip or the like. The computing unit can be part of the imaging device or designed separately from the imaging device. The imaging device can be connected to the display via the computing unit. The computing unit can be configured to generate a stereoscopic image for outputting on the display. The display can be designed as a monitor or the like. The computing unit can be connected to the control unit of the movement unit in terms of data technology and/or control technology. For example, the computing unit and the control unit can be at least partially designed as a single unit, in particular comprising common components. The computing unit can be configured to rotate the stereoscopic image before outputting it to the display, in particular as a function of the rotation parameter.

Furthermore, the invention relates to a method for operating an imaging device for stereoscopic image generation, in particular of the type described above. In one method step, in particular in a capture step, images with different selection regions can be captured. When capturing the images, a first image can be captured with a first selection region, for example at a first position of the aperture opening. The selection region, for example a position of the aperture opening, can be changed by means of the rotation of the aperture unit generated by the movement unit. At a further capture time and/or readout time via the sensor unit, a second image can be captured if the second selection region differs from the first selection region with respect to a position. By means of the computing unit, the images captured by means of the sensor unit can be combined to generate a stereoscopic image and, in particular, can be output via the display.

In a method step, in particular in a rotation step, the viewing direction of the imaging device can be changed by rotating the shaft, in particular the viewing direction unit, about the longitudinal axis. According to the method according to the invention, different selection regions for capturing images for use in stereoscopic image generation are mechanically defined from the field of view as a function of the rotation parameter with respect to the longitudinal axis. In a preferred exemplary embodiment, the selection regions can be selected as a function of the rotation parameter, so that the stereo base has a horizontal position when capturing the images for use in stereoscopic image generation. Due to such a method according to the invention, stereoscopic image generation can be reliably carried out even when the viewing direction changes. The method according to the invention makes a particularly compact configuration of the imaging device possible.

The devices and methods disclosed herein are not to be limited to the application and embodiment described above. In particular, they can have a number of individual elements, components and units as well as method steps, which differ from a number mentioned herein, in order to fulfill a function described herein. In addition, for the ranges of values specified in this disclosure, values within the stated limits shall also be deemed to be disclosed and to be usable in any manner.

It is in particular pointed out that all features and properties described with regard to a device, but also procedures, can be analogously transferred to methods in a corresponding manner and can also be used as method steps within the meaning of the invention and are considered to be disclosed as such. Likewise, method steps disclosed within the scope of the present invention description are to be regarded as device features that can be used in a device. This means that structural features mentioned in relation to methods, i.e., features relating to the device, can also be taken into account, claimed and also counted as part of the disclosure within the scope of the device claims.

### BRIEF DESCRIPTION OF THE DRAWINGS

The present invention will be described by way of example below with reference to the accompanying figures. The figures, the description, and the claims contain numerous features in combination. A person skilled in the art will also, expediently, consider the features individually and use them in combination as appropriate in the context of the claims.

If there is more than one example of any of the components described below, only one of them can be provided with a reference sign in the figures and in the description. The description of this example can be transferred accordingly to the other examples of the component. If objects are named using number words, such as first, second, third object, etc., these are used to name and/or assign objects. Accordingly, for example, a first object and a third object may be included, but not a second object. However, a number and/or sequence of objects could also be derived using number words.

In the drawings:

FIG. 1 shows a schematic representation of an imaging system comprising a display, a computing unit and an imaging device,

FIG. 2 shows a schematic cross-sectional representation of part of the imaging device in a front view,

FIG. 3 shows a schematic sequence of a method for operating the imaging device,

FIG. 4 shows a schematic representation as a cross-section of a part of an imaging device in a first alternative configuration in a front view and

FIG. 5 shows a schematic representation as a cross-section of part of an imaging device in a second alternative configuration in a front view.

### DETAILED DESCRIPTION OF EXAMPLE EMBODMENTS

FIG. 1 is an overall representation of an imaging system 46 having an imaging device 10 for stereoscopic image generation, having a computing unit 54 and having a display 56, which is designed as a monitor.

The imaging device 10 is designed as an endoscope, in particular as a stereo endoscope. The imaging device 10 comprises a shaft 12 that defines a longitudinal axis 14.

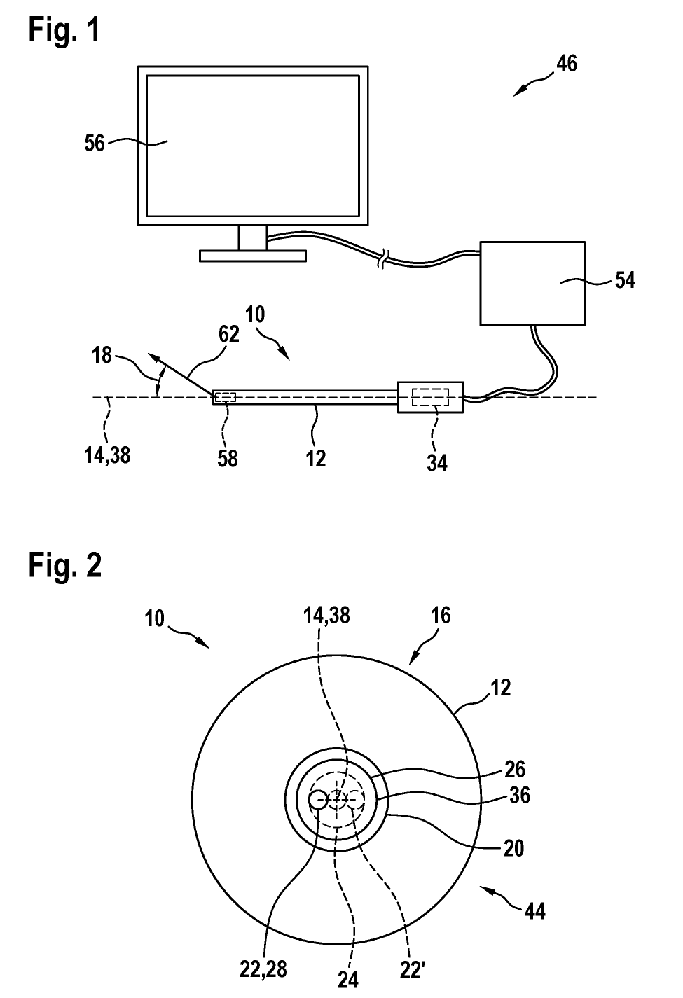

The imaging device 10 comprises a stereoscopy unit 16 for stereoscopic image generation (see FIG. 2). The imaging device 10, in particular the stereoscopy unit 16, is designed to capture images of an object point from different perspectives in order to make stereoscopic image generation possible. The computing unit 54 is configured to combine the images captured by means of the imaging device 10 from different perspectives for stereoscopic image generation. The computing unit 54 has a processor (not shown here) and a memory element (not shown here) along with an operating program stored on the memory element. The memory element is designed as a digital storage medium, for example as a memory chip or the like. The computing unit 54 is designed separately from the imaging device 10. Alternatively, the computing unit 54 can also be part of the imaging device 10. The display 56 is connected to the computing unit 54 in terms of data technology. The computing unit 54 is configured to generate a stereoscopic image for outputting on the display 56.

With reference to FIG. 2, the imaging device 10 comprises an optical unit 44 for guiding light rays from an object to be captured to a sensor unit (not shown here) of the imaging device 10. The optical unit 44 comprises at least one optical viewing direction unit 58, which defines a viewing direction 62 for viewing an examination object. The longitudinal axis 14 and the viewing direction 62 enclose an acute angle, in particular a viewing angle 18, greater than zero.

The viewing direction unit 58 can comprise a prism, a plurality of prisms or other deflecting elements for creating a viewing angle greater than 0° with respect to the longitudinal axis 14. The viewing direction unit 58 is arranged at a distal end 64 of the shaft 12. Distally before the viewing direction unit 58, a plano-concave lens can be arranged, in particular for achieving a desired imaging.

The stereoscopy unit 16 comprises a control unit (not shown here) that is configured to control the capture of images for use in stereoscopic image generation. For example, the control unit defines a capture time and/or readout time for the sensor unit. The stereoscopy unit 16 comprises a movement unit 20 for a mechanically effected change to a selection region 22, 22' from a field of view 24 of the stereoscopy unit 16 for image capture. In the exemplary embodiment shown in FIG. 2, the movement unit 20 is designed, for example, as a rotary drive. The stereoscopy unit 16 is configured to capture images at different selection regions 22, 22' for stereoscopic image generation, wherein the selection regions 22, 22' correspond in particular to images of an examination object from different perspectives.

The control unit is configured to control the movement unit 20. The control unit is part of the computing unit 54. Alternatively, the control unit can also be designed separately from the computing unit 54.

In the configuration shown in FIG. 2, the stereoscopy unit 16 comprises an aperture unit 26 comprising a mechanically positionable aperture opening 28. The aperture unit 26 comprises an aperture element 36 with the aperture opening 28. The aperture element 36 is rotatably arranged about a rotation axis 38. For example, the aperture element 36 is designed as a circular disk. The rotation axis 38 lies on the longitudinal axis 14. The aperture opening 28 is arranged eccentrically with respect to the rotation axis 38. The movement unit 20 is configured to drive the aperture element 36 to a rotation about the rotation axis 38, preferably with a constant angular velocity. The movement unit 20 comprises an electrical rotation actuator for generating the rotation of the aperture element 36 about the rotation axis 38.

The stereoscopy unit 16 is configured to define the selection region 22, 22' for capturing images for use in stereoscopic image generation as a function of a rotation parameter with respect to the longitudinal axis 14. For example, the rotation parameter determines a rotation angle of the shaft 12 and/or the stereoscopy unit 16, or of the viewing direction unit 58, about the longitudinal axis 14 and thus correlates with a viewing direction 62 of the viewing direction unit 58.

The stereoscopy unit 16 is configured to maintain a constant alignment of a stereo base when capturing images for use in stereoscopic image generation during rotations about the longitudinal axis 14. The stereo base is defined by two selection regions 22, 22', i.e. in particular, two different positions of the aperture opening 28.

The stereoscopy unit 16 comprises only a single imaging path for receiving images for stereoscopic image generation. The imaging path can be advantageously aligned with the selection region, in particular the position of the aperture opening.

The stereoscopy unit 16 comprises a position sensor system 34 for determining a value of the rotation parameter. The position sensor system 34 comprises a gyroscope, an accelerometer, an inertial measurement unit, a combination of these or the like. For example, the position sensor system 34 can capture a rotation of the shaft 12 about the longitudinal axis 14 and determine a value of a rotation angle in relation to an initial position or initial orientation of the shaft 14. In order to define the selection region for capturing images, a change relative to the initial position or relative to a previous position is captured. For example, values of the rotation angles of the shaft and/or the stereoscopy unit, or the viewing direction unit, are captured at different rotation positions. The position sensor system 34 is connected to the control unit, at least in terms of data technology.

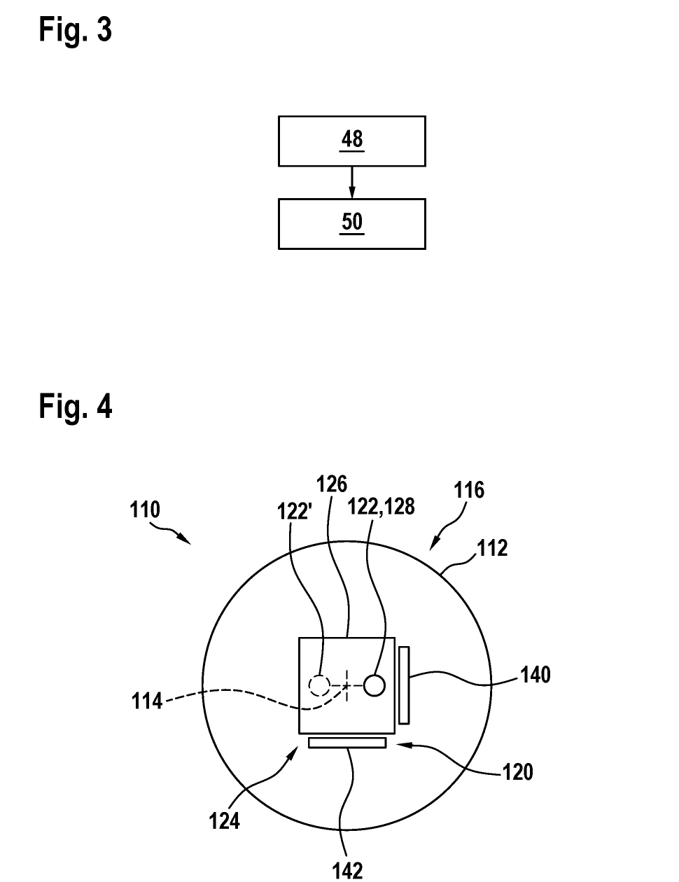

FIG. 3 shows a schematic sequence of a method for operating the imaging device 10.

In one method step, in particular in a capture step 48, images of an examination object are captured from different perspectives, i.e. with different selection regions 22, 22'. During capture of the images, a first image is captured at a first position of the aperture opening 28, i.e. at a first selection region 22. Due to the rotation of the aperture unit 26 generated by the movement unit 20, the position of the aperture opening 28 changes, so that the aperture opening 28 assumes a second position and captures a second image at a second selection region 22'. The selection regions 22 and 22' make image capture from different perspectives possible. The control unit can determine a first capture time for recording at the first selection region 22 and a second capture time for recording at the second selection region 22'. In particular, when the aperture opening 28 is rotating at a constant rotational speed about the rotation axis 38, the control unit defines a capture time in such a way that the first selection region 22 and the second selection region 22' lie on a common horizontal plane in order to keep the stereo base in horizontal alignment. The images are combined into a stereoscopic image by means of the computing unit 54 and output via the display 56. With a sufficiently high rotational speed of the aperture opening 28, approximately 30 to 60 images, or image pairs, can be captured per second. For live imaging, image capture can be carried out at approximately 120 images per second.

In one method step, in particular in a rotation step 50, the viewing direction 62 of the imaging device 10 is changed by a rotation of the shaft 12, in particular by a rotation of the viewing direction unit 58, about the longitudinal axis 14. The different selection regions 22, 22' from the field of view 24 for capturing images for use in stereoscopic image generation are defined as a function of the rotation parameter with respect to the longitudinal axis 14. The selection regions 22, 22' are selected as a function of the rotation parameter, i.e. the aperture opening 28 is positioned in such a way that the stereo base has a horizontal position when capturing the images for use in stereoscopic image generation. For this purpose, the first selection region 22 and the second selection region 22' can advantageously be opposite one another relative to the longitudinal axis 14, wherein the first position and the second position of the aperture opening 28 lie on a common horizontal with the longitudinal axis 14. The viewing direction unit 58 can thus be rotated in different viewing directions 62, which are determined by different rotation parameter values, and the stereo base for stereoscopic imaging can be kept horizontal by the stereoscopy unit according to the invention in order to achieve an optimal three-dimensional image impression.

FIG. 4 shows a sectional view of part of an imaging device 110 in a first alternative embodiment. The imaging device 110 comprises a shaft 112 that defines a longitudinal axis 114. The imaging device 110 comprises a stereoscopy unit 116 for stereoscopic image generation with a movement unit 120 for a mechanically effected change to a selection region 122, 122' from a field of view 124 of the stereoscopy unit 116 for image capture in the recording region. The stereoscopy unit 116 is configured to define the selection region 122, 122' for capturing images for use in stereoscopic image generation as a function of a rotation parameter with respect to the longitudinal axis 114.

The stereoscopy unit 116 comprises an aperture unit 126 with an aperture opening 128. A position of the aperture opening 128 defines the selection region 122, 122'. The movement unit 120 comprises a planar drive for positioning the aperture opening 128 in an aperture plane. The aperture plane runs perpendicular to the longitudinal axis 114. The planar drive is designed as a piezoelectric drive and comprises a piezoelectric X-actuator 140 and a piezoelectric Y-actuator 142, which operate in mutually orthogonal axes. The X-actuator 140 is configured to generate a movement of the aperture opening 128 in an X-direction of the aperture plane. The Y-actuator 142 is configured to generate a movement of the aperture opening 128 in a Y-direction. By means of the X-actuator 140 and the Y-actuator 142, the aperture opening 128 can be positioned at a first position for a first selection region 122 and at a second position for a second selection region 122' for different perspective image recordings in the field of view of the stereoscopy unit. As mentioned above, the first selection region 122 and the second selection region 122', or the first position and the second position of the aperture opening 28, are selected in such a way that the selection regions for stereoscopic image generation lie on a common horizontal.

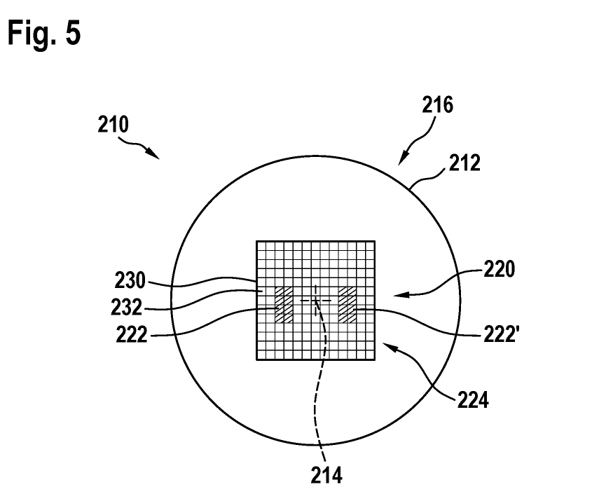

FIG. 5 shows a sectional view of part of an imaging device 210 in a second alternative embodiment. The imaging device 210 comprises a shaft 212 that defines a longitudinal axis 214. The imaging device 210 comprises a stereoscopy unit 216 for stereoscopic image generation comprising a movement unit 220 for a mechanically effected change to a selection region 222, 222' from a field of view 224 of the stereoscopy unit 216 for image capture. The stereoscopy unit 216 comprises a reflection unit 230 comprising a plurality of mechanically alignable reflection elements 232. The plurality of reflection elements 232 can together define the field of view 224, at least approximately. The stereoscopy unit 216 is configured to define the selection region 222, 222' for capturing images for use in stereoscopic image generation as a function of a rotation parameter with respect to the longitudinal axis 214. A rotation parameter value can be determined, for example, by using a position sensor system, as previously explained for the embodiments shown in FIGS. 2 and 4. The selection regions 222, 222' are defined by the alignment of the reflection elements 232. For example, the reflection elements 232 can be mechanically tilted by the movement unit 220 in such a way that light from an observation object in the selection region is fed into the imaging path. Reflection elements 232 outside the selection region do not direct any light into the imaging path. The reflection unit 230 is rotatably arranged about an axis of the optical imaging path. Alternatively, the reflection unit 230 can also be arranged in a rotationally fixed manner, for example relative to the shaft 212 and/or a viewing direction unit of the imaging device 210.

Analogous to the preceding exemplary embodiments of the stereoscopy unit according to the invention, a first selection region 222 and a second selection region 222' for different perspective image recordings can be set in the field of view of the stereoscopy unit by means of the reflection unit 230. Advantageously, the first selection region 222 and the second selection region 222', or the associated alignment of the reflection elements 232, are selected in such a way that the selection regions for stereoscopic image generation lie on a common horizontal.

### LIST OF REFERENCE SIGNS

10, 110, 210 Imaging device

12, 112, 212 Shaft

14, 114, 214 Longitudinal axis

16, 116, 216 Stereoscopy unit

18 Viewing angle

20, 120, 220 Movement unit

22, 122, 222 Selection region

24, 124, 224 Field of view

26, 126 Aperture unit

28, 128 Aperture opening

230 Reflection unit

232 Reflection elements

34 Position sensor system

36 Aperture element

38 Rotation axis

140 X-actuator

142 Y-actuator

44 Optical unit

46 Imaging system

48 Capture step

50 Rotation step

54 Computing unit

56 Display

58 Viewing direction unit

62 Viewing direction

64 Distal end

1-15. (canceled).

16. An imaging device, in particular an endoscope device, comprising:
a shaft that defines a longitudinal axis, and
a stereoscopy unit for stereoscopic image generation,
wherein the stereoscopy unit comprises a movement unit for a mechanically causing a change to a selection area within a field of view of the stereoscopy unit for image capture, and
wherein the stereoscopy unit is configured to define the selection area for capturing images for use in stereoscopic image generation depending on a rotation parameter with respect to the longitudinal axis.

17. The imaging device according to claim 16, wherein the stereoscopy unit comprises an aperture unit with at least one mechanically positionable aperture opening, and
wherein the stereoscopy unit is configured to define a position of the aperture opening for capturing images for use in stereoscopic image generation depending on a rotation parameter with respect to the longitudinal axis.

18. The imaging device according to claim 16, wherein the stereoscopy unit comprises a reflection unit with mechanically alignable reflection elements, wherein the stereoscopy unit is configured to define an alignment of the reflection elements for capturing images for use in stereoscopic image generation depending on a rotation parameter with respect to the longitudinal axis.

19. The imaging device according to claim 16, wherein the stereoscopy unit is configured to capture images with different selection areas for stereoscopic image generation, in particular at different positions of an aperture opening of an aperture unit with at least one mechanically positionable aperture opening or at different alignments of reflection elements of a reflection unit with mechanically alignable reflection elements.

20. The imaging device according to claim 16, wherein the stereoscopy unit is configured to maintain a constant alignment of a stereo base defined by the selection area, when capturing images for use in stereoscopic image generation, during rotations about the longitudinal axis.

21. The imaging device according to claim 17, wherein the movement unit comprises a planar drive for translating the aperture opening in a plane perpendicular to the longitudinal axis, wherein the planar drive is configured in such a way that it positions the aperture opening as a function of the rotation parameter.

22. The imaging device according to claim 21, wherein the planar drive is a piezoelectric drive with an X-actuator and a Y-actuator.

23. The imaging device at least according to claim 17, wherein the aperture unit comprises an aperture element with the aperture opening, which is rotatably arranged about a rotation axis.

24. The imaging device according to claim 23, wherein the movement unit is configured to drive the aperture element to a rotation about the rotation axis at a constant angular velocity.

25. The imaging device according to claim 17, wherein the aperture opening is arranged eccentrically with respect to the rotation axis.

26. The imaging device according to claim 16, wherein the stereoscopy unit comprises only a single imaging path for receiving the images for stereoscopic image generation.

27. The imaging device according to claim 16, wherein the stereoscopy unit comprises a position sensor system for determining the rotation parameter.

28. The imaging device according to claim 16, comprising an optical viewing direction unit that defines a viewing direction for viewing an examination object, wherein the longitudinal axis and the viewing direction enclose an acute angle greater than zero.

29. A method for operating an imaging device for stereoscopic image generation, wherein the imaging device comprises a shaft that defines a longitudinal axis, and wherein different selection areas for capturing images for use in stereoscopic image generation are mechanically determined from a field of view as a function of a rotation parameter with respect to the longitudinal axis.

30. An imaging device, in particular an endoscope device, comprising:
a shaft that defines a longitudinal axis, and
a stereoscopy unit for stereoscopic image generation,
wherein the stereoscopy unit comprises an aperture unit with at least one of a mechanically positionable aperture opening or a mechanically alignable reflection element, and
wherein the stereoscopy unit is configured to define a position of the aperture opening or an alignment of the reflection elements for capturing images for use in stereoscopic image generation depending on a rotation parameter with respect to the longitudinal axis.

31. The imaging device according to claim 30, wherein
wherein the stereoscopy unit comprises the reflection unit with mechanically alignable reflection elements, and
wherein the stereoscopy unit is configured to define the alignment of the reflection elements for capturing images for use in stereoscopic image generation depending on the rotation parameter with respect to the longitudinal axis.

32. The imaging device according to claim 30, wherein
wherein the stereoscopy unit comprises the mechanically positionable aperture opening, and
wherein the stereoscopy unit is configured to define the position of the aperture opening for capturing images for use in stereoscopic image generation depending on the rotation parameter with respect to the longitudinal axis.
